(19) United States
(12) Patent Application Publication
(10) Pub. No.: US 2022/0354972 A1
(43) Pub. Date: Nov. 10, 2022

(54) IN SITU RECRUITMENT, REPROGRAMMING, AND RELEASE OF CAR-T CELLS

(71) Applicants: NORTH CAROLINA STATE UNIVERSITY, Raleigh, NC (US); THE UNIVERSITY OF NORTH CAROLINA AT CHAPEL HILL, chapel Hill, NC (US)

(72) Inventors: Yevgeny BRUDNO, Cary, NC (US); Gianpietro DOTTI, Chapel Hill, NC (US); Pritha AGARWALLA, Raleigh, NC (US)

(21) Appl. No.: 17/621,303

(22) PCT Filed: Jun. 18, 2020
(86) PCT No.: PCT/US2020/038386

§ 371 Date: Dec. 21, 2021

Related U.S. Application Data
(60) Provisional application No. 62/864,703, filed on Jun. 21, 2019.

(51) Int. Cl. A61K 49/18; C07K 14/725; C12N 5/0783; C12N 15/86

(52) U.S. Cl. CPC A61K 49/1803; C07K 14/7051; C12N 5/0636; C12N 15/86; A61K 2039/5156

(57) ABSTRACT

Disclosed are hydrogel matrixes for use in recruitment and reprogramming of CAR T cells, CAR NK cells, CAR NK T cells, CAR macrophage, Tumor infiltrating NK cells, tumor infiltrating lymphocytes, and marrow infiltrating lymphocytes.

### I. BACKGROUND

Adoptive transfer of chimeric antigen receptor (CAR)-redirected T lymphocytes (CAR-T cells) produces impressive clinical responses against B-cell malignancies and are effective for other cancer types. Current approaches for clinical-scale manufacturing of CAR-T cells require extensive cell manipulation ex vivo: isolation of autologous T cells, transduction with CAR-encoding viral vectors, and CAR-T cell expansion ex vivo before infusion back into the patient. The elaborate and time intensive ex vivo procedures come with substantial costs: the procedure takes 3-4 weeks and costs approximately S500,000. These costs limit the potential of expanding this technology to other cancers and to patients. What are needed are new ways to recruit and modify T cells that avoid the problems associated with the present CAR T cells.

### II. SUMMARY

Disclosed are methods and compositions related to hydrogels comprises chemoattractants.

In one aspect, disclosed herein are hydrogel matrixes comprising one or more chemoattractants, wherein the one or more chemoattractants comprise C-C motif chemokine ligand (CCL) 1 (CCL1), CCL5, CCL19, CCL21, CCL22, CCL28, C-X-C Motif Chemokine Ligand (CXCL) 1 (CXCL1), CXCL9, CXCL10, CXCL11, CXCL12, M-CSF, GM-CSF, MCP-1, MCP-3, CCL2, CCL3, CCL7, CCL20, CX3CL1, BRAK, IL-12, S1P, and/or MCP2.

Also disclosed herein are hydrogel matrixes of any preceding aspect, further comprising a viral vector (such as, for example, a lentivirus, retrovirus, adenovirus, or adeno-associated virus) encoding a chimeric antigen receptor (CAR), NK cell receptor (including, but not limited to anti-CD3 antibody, CD1d, an Fc fragment of an immunoglobulin, or anti-Fc gamma receptor (FcγRIII) antibody), NK T cell receptor and/or T cell receptor (such as an antigens-specific T cell receptor).

In one aspect, also disclosed herein are hydrogel matrixes of any preceding aspect, further comprising one or more antibodies, cytokines, and/or co-stimulatory molecules which activate a T cell, natural killer (NK) cell, NK T cell, tumor infiltrating lymphocyte (TIL), a marrow infiltrating lymphocyte (MIL), tumor infiltrating NK cell (TINK), dendritic cells, or a macrophage. For example, the antibody can comprises anti-CD3, anti-CD28, anti-Inducible Costimulator (ICOS), anti-CD40L, anti-DAP10; and the cytokine can comprise IL-2, IL-7, IL-15, IL-21, TNF-α, or IFN-γ.

Also disclosed herein are hydrogel matrixes of any preceding aspect, further comprising a chemotherapeutic agent.

In one aspect, disclosed herein are methods of treating, preventing, inhibiting, and/or reducing a cancer or metastasis in a subject comprising administering to the subject the hydrogel matrix of any preceding aspect. For example, disclosed herein are methods of treating a cancer in a subject comprising administering to the subject a hydrogel matrix comprising one or more chemoattractants, wherein the one or more chemoattractants comprise C-C motif chemokine ligand (CCL) 1 (CCL1), CCL5, CCL19, CCL21, CCL22, CCL28, C-X-C Motif Chemokine Ligand (CXCL) 1 (CXCL1), CXCL9, CXCL10, CXCL11, CXCL12, M-CSF, GM-CSF, MCP-1, MCP-3, CCL2, CCL3, CCL7, CCL20, CX3CL1, BRAK, IL-12, S1P, and/or MCP2; and wherein the chemoattractant attracts and retains an immune cell (such as, for example, a T cell, NK cell, NK T cell, TIL, MIL, TINK, dendritic cell, and/or macrophage) to the hydrogel.

Also disclosed herein are methods of treating, preventing, inhibiting, and/or reducing a cancer or metastasis of any preceding aspect, wherein the hyrodrogel matrix further comprises an immune blockade inhibitor and/or a chemotherapeutic agent.

In one aspect, disclosed herein are methods of treating, preventing, inhibiting, and/or reducing a cancer or metastasis of any preceding aspect, wherein the hydrogel further comprises a viral vector encoding a chimeric antigen receptor (CAR), NK cell receptor (including, but not limited to anti-CD3 antibody, CD1d, an Fc fragment of an immunoglobulin, or anti-Fc gamma receptor (FcγRIII) antibody), NK T cell receptor, and/or T cell receptor (TCR) (including, antigen-specific T cell receptor). In one aspect, the viral vector transduces the immune cell; and the transduced immune cell is released from the hydrogel to the cancer. It is understood and herein contemplated that the viral vector can be introduced into the hydrogel matrix prior to administration to the subject or from about 1 day to about 14 days following administration of the hydrogel matrix to the subject. That is, the viral vector can be introduced into the hydrogel in vivo.

Also disclosed herein are methods of treating, preventing, inhibiting, and/or reducing a cancer or metastasis of any preceding aspect, wherein the immune cell is released from about 1 week to about 12 weeks after administration of the hydrogel.

In one aspect, disclosed herein are methods of treating, preventing, inhibiting, and/or reducing a cancer or metastasis of any preceding aspect, wherein the one or more chemoattractants are released from about 1 hour after administration of the hydrogel to about 12 weeks after administration of the hydrogel.

In one aspect, disclosed herein are methods of transducing an immune cell (such as, for example, T cell, NK cell, NK T cell, TIL, or MIL) in a subject, the method comprising administering to the subject a hydrogel comprising one or more chemoattractants (such as, for example, CCL1, CCL5, CCL19, CCL21, CCL22, CCL28, CXCL1, CXCL9, CXCL10, CXCL11, CXCL12, M-CSF, GM-CSF, MCP-1, MCP-3, CCL2, CCL3, CCL7, CCL20, CX3CL1, BRAK, IL-12, S1P, and/or MCP2) and a viral vector (such as a lentivirus, retrovirus, adenovirus, or adeno-associated virus) encoding a transgene (such as, for example, a CAR, NK cell receptor (including, but not limited to anti-CD3 antibody, CD1d, an Fc fragment of an immunoglobulin, or anti-Fc gamma receptor (FcγRIII) antibody), NK T cell receptor, and/or TCR (including, but not limited to antigen specific TCR)). It is understood and herein contemplated that the viral vector can be introduced into the hydrogel matrix prior to administration to the subject or from about 1 day to about 14 days following administration of the hydrogel matrix to the subject. That is, the viral vector can be introduced into the hydrogel in vivo.

Also disclosed herein are methods of transducing an immune cell of any preceding aspect, wherein the one or more chemoattractants are released from about 1 hour after administration of the hydrogel to about 12 weeks after administration of the hydrogel.

In one aspect, disclosed herein are methods of transducing an immune cell of any preceding aspect, wherein the hydrogel further comprises one or more antibodies, cytokines, and/or co-stimulatory molecules which activate a T cell, NK cell, NK T cell, TIL, or MIL. For example, the antibody can comprises anti-CD28, CD3, B7-1, B7-2, anti-inducible costimulator (ICOS), ICOS ligand, anti-CD27, CD70, 4-1BBL, anti-41-BB, anti-CD40L, CD40, anti-DAP10, anti-CD30, CD30L, anti-TIM-1, anti-TIM-2, anti-TIM-3, anti-CD44, anti-NK1.1, lectin like transcript-1 (LLT-1), anti-CD137, CD48, MICA, anti-2B4, and anti-glucocorticoid-induced tumor necrosis factor receptor related protein (GITR); and the cytokine can comprise IL-2, IL-7, IL-15, IL-21, TNF-α, or IFN-γ.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 is a representation of the generation of CAR-T cells in situ. Implanted scaffolds release chemokine to recruit host T cells. Viral vectors reprogram T cells with tumor-specific CAR constructs and CAR-modified T cells migrate out to fight cancer.

FIG. 2 shows a representation of making macroporous gels via cryogelation

FIG. 3 shows SEM pictures of gels prepared by freeze dry regimen (−20° C.).

FIGS. 4A and 4B show that CXCL10 mediates T-cell recruitment to implanted scaffolds. FIG. 4A shows the experimental timeline and FIG. 4B shows FACS analysis of CD3+ T cells recruited to blank (control) or CXCL10-releasing scaffolds.

FIGS. 5A, 5B, 5C, and 5D show pre-seeded and recruited T-cells can be transduced inside of implanted scaffolds. Experimental timelines (5A, 5C) and FACS analysis (5B, 5D) for GFP+ T cells pre-seeded into scaffolds or recruited to scaffold by CXCL10. D shows additional impact of CD3/CD28 and IL-2 in scaffolds.

FIG. 6 shows the in situ generation of CAR T cells.

FIG. 7 shows in vivo transduction of recruited T cells with CD19 CAR virus.

FIG. 8 shows a schematic representation of the creation of a mouse Burkitt's lymphoma tumor model.

FIG. 9A shows luminescence images of tumor size at 19, 24, 29, 33, and 43 days post tumor inoculation in animals receiving no treatment, CD19 CAR T cells i.v., or CCI-alginate scaffolds (CCI-Alg).

FIG. 9B shows the total flux and percentage body weight change at various time points post tumor inoculation in animals receiving no treatment, CD19 CAR T cells i.v., or CCI-alginate scaffolds (CCI-Alg).

FIG. 10 shows the number CAR+ cells per 100 ul of blood in mice implanted with CCI scaffolds or i.v. infused with CAR-T cells. Mice were bled via cheek bleed, red blood cells were lysed, cells were stained with Hu-CD45, Hu-CD3 and CAR.19 antibodies and analyzed by flow cytometry.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

In this specification and in the claims which follow, reference will be made to a number of terms which shall be defined to have the following meanings:

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration, but are undetectable or detectable at negligible amounts in distal parts of the subject's body. Administration includes self-administration and the administration by another.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “control” is an alternative subject or sample used in an experiment for comparison purposes. A control can be “positive” or “negative.”

“Controlled release” or “sustained release” refers to release of an agent from a given dosage form in a controlled fashion in order to achieve the desired pharmacokinetic profile in vivo. An aspect of “controlled release” agent delivery is the ability to manipulate the formulation and/or dosage form in order to establish the desired kinetics of agent release.

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant. Increases can also be referenced in terms of fold increases. For example, an increase can comprise a 2, 3, 4, 5, 6, 7, 8, 9, 10, 15, 20, 30, 40, 50, 60, 70, 80, 90, 100, 200, 300, 400, 500, 600, 700, 800, 900, 10³, 10⁴, 10⁵, 10⁶, 10⁷, 10⁸, 10⁹, 10¹⁰-fold increase.

A “decrease” can refer to any change that results in a smaller gene expression, protein expression, amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant. Decreases can also be referenced in terms of fold decreases. For example, an decrease can comprise a 2, 3, 4, 5, 6, 7, 8, 9, 10, 15, 20, 30, 40, 50, 60, 70, 80, 90, 100, 200, 300, 400, 500, 600, 700, 800, 900, 10³, 10⁴, 10⁵, 10⁶, 10⁷, 10⁸, 10⁹, 10¹⁰-fold decrease.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The terms “prevent,” “preventing.” “prevention,” and grammatical variations thereof as used herein, refer to a method of partially or completely delaying or precluding the onset or recurrence of a disease and/or one or more of its attendant symptoms or barring a subject from acquiring or reacquiring a disease or reducing a subject's risk of acquiring or reacquiring a disease or one or more of its attendant symptoms.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular hydrogel matrix comprising a chemoattract is disclosed and discussed and a number of modifications that can be made to a number of molecules including the hydrogel matrix comprising a chemoattract are discussed, specifically contemplated is each and every combination and permutation of hydrogel matrix comprising a chemoattract and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Chimeric Antigen Receptor (CAR) immunotherapy therapy is a type of cancer immunotherapy that involves genetic modification of patient's autologous T-cells, NK cells, and/or macrophage into CAR-T cells, CAR-NK cells, CAR-NK-T cells, or CAR Macrophage (CARMA), respectively. These CAR cells express antibody based chimeric antigen receptor that can target a specific tumor antigen. The process involves isolation of autologous T cells, NK cells, NK T cells, or macrophage, transduction with CAR-encoding viral vectors, and CAR-T cell, CAR-NK cell, CAR-NK-T cell, or CARMA expansion ex vivo before infusion back into the patient. Despite remarkable clinical success, the laborious, time consuming and costly procedures associated with generating clinical grade CAR immune cells (such as, for example, CAR T cells) in accordance with stringent regulatory standards remains a major obstacle for implementing CAR immune cell therapy as a standard-of-care in the treatment of cancer. Approaches that can eliminate the time and costs associated with generating CAR immune cells cells is of huge clinical significance and could dramatically expand the availability of CAR T cell, CAR-NK cell, CAR-NK-T cell, and/or CARMA therapy.

Generating tumor-specific CAR immune cells in situ can overcome many challenges that have been associated with ex vivo generation of CAR-T cells. Chimeric antigen receptors (CARs) are synthetic receptors that re-direct immune cell (such as, for example, CAR T cell, CAR-NK cell, CAR-NK-T cell, or CARMA) specificity towards tumor-associated antigens. Infusion of CD19. CAR-T cells shows remarkable antitumor effects in clinical trials with the first CD19. CAR-T therapy approved by the FDA in 2017. T cells collection, ex vivo activation, genetic modification and expansion of the cells, and subsequent infusion into patients is both time consuming (˜4 weeks) and expensive (—S500,000). Given the burden cancer already poses to the healthcare system, providing CAR immune cell therapy to millions of cancer patients presents an immense challenge. Herein is shown that CAR immune cells can be generated in situ in a macroporous scaffold loaded with a T-cell recruitment factor and CAR-encoding viral vectors. This approach eliminates the time and costs associated with generating CAR immune cells (such as, for example, CAR T cell, CAR-NK cell, CAR-NK-T cell, or CARMA) can be of huge clinical significance and can dramatically expand the availability of CAR immune cell therapy.

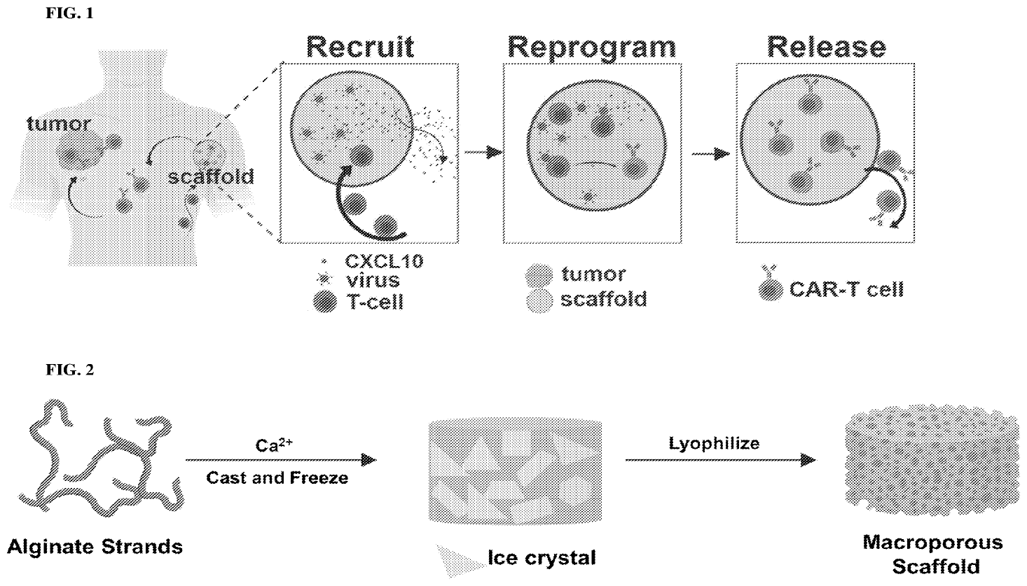

Disclosed herein is a strategy for in situ generation of CAR immune cells inside of patients using a bioactive macroporous scaffold. The scaffold recruits the immune cells, activates and reprograms them into CAR immune cells, and releases the CAR immune cells into the body to home to tumors (FIG. 1). Controlled release of chemokines from the scaffold creates a gradient that attracts T cells, (including, but not limited to TILs and MILs) NK cells (including, but not limited to TINKs), NK T cells, dendritic cells, and/or macrophage to the scaffold. The macroporous scaffold promotes chemokine-mediated T cell, NK cell, NK T cell, dendritic cell, and/or macrophage infiltration and together with embedded cytokines and antibodies promotes the survival and expansion of T cells, NK cells, NK T cells, dendritic cell, and/or macrophage and provides an interface for transduction by viral particles. Once the chemokine is completely released, its depletion promotes CAR-T cell, CAR-NK cell, CAR-NK-T cell, or CARMA migration out of the scaffold. In this approach, T cells, NK cells, NK T cells, dendritic cells, and/or macrophage are recruited to the biomaterial scaffold, reprogrammed with the CAR-gene by viral vectors, and released from the scaffold into circulation, leading to seamless generation of CAR-T cells, CAR-NK cells, CAR-NK-T cells, or CARMA in situ. Accordingly, in one aspect disclosed herein are hydrogel matrixes comprising one or more chemoattractants.

It is understood and herein contemplated that the purpose of the chemoattractant to is attract an immune cell (such as, for example, a T cell, natural killer (NK) cell, NK T cell, tumor infiltrating lymphocyte (TIL), marrow infiltrating lymphocyte (MIL), tumor infiltrating NK cell (TINK), dendritic cells, and/or macrophage) to the hydrogel. Examples of the one or more chemoattractants for use in the disclosed hydrogels, include, but are not limited to C-C motif chemokine ligand (CCL) 1 (CCL1), CCL5. CCL19, CCL21, CCL22, CCL28, C-X-C Motif Chemokine Ligand (CXCL) 1 (CXCL1), CXCL9, CXCL10, CXCL11, CXCL12, M-CSF, GM-CSF, MCP-1, MCP-3, CCL2, CCL3, CCL7, CCL20, CX3CL1, BRAK, IL-12, S1P, and/or MCP2.

The hydrogels disclosed herein can be made using any suitable biodegradable polymer. “Polymer” refers to a relatively high molecular weight organic compound, natural or synthetic, whose structure can be represented by a repeated small unit, the monomer. Non-limiting examples of polymers include polyethylene, rubber, cellulose. Synthetic polymers are typically formed by addition or condensation polymerization of monomers. The term “copolymer” refers to a polymer formed from two or more different repeating units (monomer residues). By way of example and without limitation, a copolymer can be an alternating copolymer, a random copolymer, a block copolymer, or a graft copolymer. It is also contemplated that, in certain aspects, various block segments of a block copolymer can themselves comprise copolymers. The term “polymer” encompasses all forms of polymers including, but not limited to, natural polymers, synthetic polymers, homopolymers, heteropolymers or copolymers, addition polymers, etc.

In one aspect, the hydrogel can comprise a biocompatible polymer (such as, for example, alginate). Such polymers can also serve to slowly release CAR T cell, CAR NK cell, CAR NK T cell, CARMA, TIL, MIL, TINK, and/or MIL into the tissue. As used herein biocompatible polymers include, but are not limited to polysaccharides such as alginate, chitosan, hyaluronic acid; hydrophilic polypeptides; proteins such as collagen, fibrin, and gelatin; poly(amino acids) such as poly-L-glutamic acid (PGS), gamma-polyglutamic acid, poly-L-aspartic acid, poly-L-serine, or poly-L-lysine; polyalkylene glycols and polyalkylene oxides such as polyethylene glycol (PEG), polypropylene glycol (PPG), and poly(ethylene oxide) (PEO); poly(oxyethylated polyol); poly(olefinic alcohol); polyvinylpyrrolidone); poly(hydroxyalkylmethacrylamide); poly(hydroxyalkylmethacrylate); poly(saccharides); poly(hydroxy acids); poly(vinyl alcohol), polyhydroxyacids such as poly(lactic acid), poly (gly colic acid), and poly (lactic acid-co-glycolic acids); polyhydroxyalkanoates such as poly3-hydroxybutyrate or poly4-hydroxybutyrate; polycaprolactones; poly(orthoesters); polyanhydrides; poly(phosphazenes); poly(lactide-co-caprolactones); polycarbonates such as tyrosine polycarbonates; polyamides (including synthetic and natural polyamides), polypeptides, and poly(amino acids); polyesteramides; polyesters; poly(dioxanones); poly(alkylene alkylates); hydrophobic polyethers; polyurethanes; polyetheresters; polyacetals; polycyanoacrylates; polyacrylates; polymethylmethacrylates; polysiloxanes; poly(oxyethylene)/poly(oxypropylene) copolymers; polyketals; polyphosphates; polyhydroxyvalerates; polyalkylene oxalates; polyalkylene succinates; poly(maleic acids), as well as copolymers thereof. Biocompatible polymers can also include polyamides, polycarbonates, polyalkylenes, polyalkylene glycols, polyalkylene oxides, polyalkylene terepthalates, polyvinyl alcohols (PVA), methacrylate PVA (m-PVA), polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly (methyl methacrylate), poly(ethylmethacrylate), poly(butylmethacrylate), poly(isobutylmethacrylate), poly(hexlmethacrylate), poly(isodecylmethacrylate), poly(lauryl methacrylate), poly (phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), poly(vinyl acetate, poly vinyl chloride polystyrene and polyvinylpryrrolidone, derivatives thereof, linear and branched copolymers and block copolymers thereof, and blends thereof. Exemplary biodegradable polymers include polyesters, poly(ortho esters), poly(ethylene amines), poly(caprolactones), poly(hydroxybutyrates), poly(hydroxyvalerates), polyanhydrides, poly(acrylic acids), polyglycolides, poly(urethanes), polycarbonates, polyphosphate esters, polyphospliazenes, derivatives thereof, linear and branched copolymers and block copolymers (including triblock copolymers) thereof, and blends thereof.

In some embodiments the particle contains biocompatible and/or biodegradable polyesters or polyanhydrides such as poly(lactic acid), poly(glycolic acid), and poly(lactic-co-glycolic acid). The particles can contain one more of the following polyesters: homopolymers including glycolic acid units, referred to herein as “PGA”, and lactic acid units, such as poly-L-lactic acid, poly-D-lactic acid, poly-D,L-lactic acid, poly-L-lactide, poly-D-lactide, and poly-D,L-lactide5 collectively referred to herein as “PLA”, and caprolactone units, such as poly(e-caprolactone), collectively referred to herein as “PCL”; and copolymers including lactic acid and glycolic acid units, such as various forms of poly(lactic acid-co-glycolic acid) and poly(lactide-co-glycolide) characterized by the ratio of lactic acid:glycolic acid, collectively referred to herein as “PLGA”; and polyacrylates, and derivatives thereof. Exemplary polymers also include copolymers of polyethylene glycol (PEG) and the aforementioned polyesters, such as various forms of PLGA-PEG or PLA-PEG copolymers, collectively referred to herein as “PEGylated polymers”. In certain embodiments, the PEG region can be covalently associated with polymer to yield “PEGylated polymers” by a cleavable linker. In one aspect, the polymer comprises at least 60, 65, 70, 75, 80, 85, 89, 90, 91, 92, 93, 94, 95, 96, 97, 98, or 99 percent acetal pendant groups.

The triblock copolymers disclosed herein comprise a core polymer such as, example, polyethylene glycol (PEG), polyvinyl acetate, polyvinyl alcohol, polyvinyl pyrrolidone (PVP), polyethyleneoxide (PEO), poly(vinyl pyrrolidone-co-vinyl acetate), polymethacrylates, polyoxyethylene alkyl ethers, polyoxyethylene castor oils, polycaprolactam, polylactic acid, polyglycolic acid, poly(lactic-glycolic) acid, poly(lactic co-glycolic) acid (PLGA), cellulose derivatives, such as hydroxymethylcellulose, hydroxypropylcellulose and the like.

In one aspect, the hydrogel matrixes can further comprise an immune activating and/or sustaining antibodies, chemokines, and cytokines such as, for example, CD28, CD3, IL-2, IL-7, IL-15, IL-21, IFN-γ, and TNF-α.

It is understood and herein contemplated that the disclosed hydrogel matrixes can further comprise one or more immune checkpoint inhibitors and/or chemotherapeutic agents. Chemotherapeutic agents that can be used in the disclosed hydrogel matrixes can comprise any chemotherapeutic known in the art, the including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPDX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNIS ONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome). Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). Checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

In one aspect, also disclosed herein are hydrogel matrixes as disclosed herein, further comprising one or more antibodies, cytokines, and/or co-stimulatory molecules which activate a T cell, natural killer (NK) cell, NK T cell, dendritic cell, macrophage, tumor infiltrating NK cell (TINK), tumor infiltrating lymphocyte (TIL), or a marrow infiltrating lymphocyte (MIL). For example, the antibody can comprises anti-CD28, CD3, B7-1, B7-2, anti-inducible costimulator (ICOS), ICOS ligand, anti-CD27, CD70, 4-1BBL, anti-41-BB, anti-CD40L, CD40, anti-DAP10, anti-CD30, CD30L, anti-TIM-1, anti-TIM-2, anti-TIM-3, anti-CD44, anti-NK1.1, lectin like transcript-1 (LLT-1), anti-CD137, CD48, MICA, anti-2B4, and anti-glucocorticoid-induced tumor necrosis factor receptor related protein (GITR); and the cytokine can comprise IL-2, IL-7, IL-15, IL-21, TNF-α, or IFN-γ.

One advantage of the disclosed hydrogels is that once the immune cell is brought into contact with the hydrogel, the immune cell can be transduced to comprise a transgene such as a chimeric antigen receptor (for example, an antibody or scFv that targets a tumor specific antigen), T cell receptor (such as antigen-specific T cell receptor), NK cell receptor (including, but not limited to anti-CD3 antibody, CD1d, an Fc fragment of an immunoglobulin, or anti-Fc gamma receptor (FcγRIII) antibody), NK T cell receptor. Tumor antigens are proteins that are produced by tumor cells that elicit an immune response, particularly T-cell mediated immune responses. The additional antigen binding domain can be an antibody, or a natural ligand of the tumor antigen, or a molecule that recognizes peptides derived from the tumor antigen presented by MHC molecules. The selection of the additional antigen binding domain will depend on the particular type of cancer to be treated. Tumor antigens are well-known in the art and include, for example, a glioma-associated antigen, carcinoembryonic antigen (CEA), EGFRvIII, IL-11Ra, IL-13Ra, EGFR, FAP, B7H3, Kit, CA LX, CS-1, MUC1, BCMA, bcr-abl, HER2, β-human chorionic gonadotropin, alphafetoprotein (AFP), ALK, CD19, CD123, cyclin Bl, lectin-reactive AFP, Fos-related antigen 1, ADRB3, thyroglobulin, EphA2, RAGE-1, RU1, RU2, SSX2, AKAP-4, LCK, OY-TES1, PAX5, SART3, CLL-1, fucosyl GM1, GloboH, MN-CA IX, EPCAM, EVT6-AML, TGS5, human telomerase reverse transcriptase, plysialic acid, PLAC1, RU1, RU2 (AS), intestinal carboxyl esterase, lewisY, sLe, LY6K, mut hsp70-2, M-CSF, MYCN, RhoC, TRP-2, CYPIBI, BORIS, prostase, prostate-specific antigen (PSA), PAX3, PAP, NY-ESO-1, LAGE-la, LMP2, NCAM, p53, p53 mutant, Ras mutant, gp100, prostein, OR51E2, PANX3, PSMA, PSCA, Her2/neu, hTERT, HMWMAA, HAVCR1, VEGFR2, PDGFR-beta, survivin and telomerase, legumain, HPV E6, E7, sperm protein 17, SSEA-4, tyrosinase, TARP, WT1, prostate-carcinoma tumor antigen-1 (PCTA-1), ML-IAP, MAGE, MAGE-A1, MAD-CT-1, MAD-CT-2, MelanA/MART 1, XAGE1, ELF2M, ERG (TMPRSS2 ETS fusion gene), NA17, neutrophil elastase, sarcoma translocation breakpoints, NY-BR-1, ephnnB2, CD20, CD22, CD24, CD30, CD33, CD38, CD44v6, CD97, CD171, CD179a, androgen receptor, FAP, insulin growth factor (IGF)-I, IGFII, IGF-I receptor, GD2, o-acetyl-GD2, GD3, GM3, GPRC5D, GPR20, CXORF61, folate receptor (FRa), folate receptor beta, ROR1, Flt3, TAG72, TN Ag, Tie 2, TEM1, TEM7R, CLDN6, TSHR, UPK2, and mesothelin.

Delivery of the Compositions to Cells

Transduction of the immune cell can occur by any means known in the art. In one aspect, transduction of the immune cell can occur via a viral vector encoding a transgene (such as, for example a CAR). Accordingly, in one aspect, disclosed herein are any of the hydrogels disclosed, further comprising a viral vector (such as, for example, a lentivirus, retrovirus, adenovirus, or adeno-associated virus) encoding a transgene, such as, for example, a chimeric antigen receptor (CAR), T cell receptor, NK cell receptor, and/or NK T cell receptor.

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

### (1) Retroviral Vectors

A retrovirus is an animal virus belonging to the virus family of Retroviridae, including any types, subfamilies, genus, or tropisms. Retroviral vectors, in general, are described by Verma, I. M., Retroviral vectors for gene transfer.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically, a retroviral genome, contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

### (2) Adenoviral Vectors

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

### (3) Adeno-Associated Viral Vectors

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19 (such as, for example at AAV integration site 1 (AAVS1)). Vectors which contain this site-specific integration property are preferred. An especially preferred embodiment of this type of vector is the P4.1 C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The disclosed vectors thus provide DNA molecules which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

### (4) Large Payload Viral Vectors

Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson, Curr Opin Mol Ther 5: 633-644, 1999). These large DNA viruses (herpes simplex virus (HSV) and Epstein-Barr virus (EBV), have the potential to deliver fragments of human heterologous DNA >150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA >220 kb and to infect cells that can stably maintain DNA as episomes.

Other useful systems include, for example, replicating and host-restricted non-replicating vaccinia virus vectors.

Expression Systems

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

a) Viral Promoters and Enhancers

Preferred promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis Enhancers f unction to increase transcription from nearby promoters Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promotor and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

It has been shown that all specific regulatory elements can be cloned and used to construct expression vectors that are selectively expressed in specific cell types such as melanoma cells. The glial fibrillary acetic protein (GFAP) promoter has been used to selectively express genes in cells of glial origin.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases. It is also preferred that the transcribed units contain other standard sequences alone or in combination with the above sequences improve expression from, or stability of, the construct.

b) Markers

The viral vectors can include nucleic acid sequence encoding a marker product. This marker product is used to determine if the gene has been delivered to the cell and once delivered is being expressed. Preferred marker genes are the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. T. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles. pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid. pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines

b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

### C. Methods of Transducing an Immune Cell

It is understood and herein contemplated that the disclosed hydrogels can be used to attract immune cells (such as, for example, T cell, NK cell, NK T cell, dendritic cell, macrophage, TINK, TIL, and/or MIL) to the hydrogel where in vivo transduction of the immune cell can take place. In one aspect, disclosed herein are methods of transducing an immune cell (such as, for example, T cell, NK cell, NK T cell, dendritic cell, macrophage, TINK, TIL, or MIL) in a subject, the method comprising administering to the subject a hydrogel comprising one or more chemoattractants (such as, for example, CCL1, CCL5, CCL19, CCL21, CCL22, CCL28, CXCL1, CXCL9, CXCL10, CXCL11. CXCL12, M-CSF, GM-CSF, MCP-1, MCP-3, CCL2, CCL3, CCL7, CCL20, CX3CL1, BRAK, IL-12, SIP, and/or MCP2) and a viral vector (such as a lentivirus, retrovirus, adenovirus, or adeno-associated virus) encoding a transgene (such as, for example, a CAR, NK cell receptor (including, but not limited to anti-CD3 antibody, CD1d, an Fc fragment of an immunoglobulin, or anti-Fc gamma receptor (FcγRIII) antibody), T cell receptor (such as an antigen specific T cell receptor), or NK T cell receptor).

It is understood and herein contemplated that the viral vector can be introduced into the hydrogel matrix prior to administration to the subject or from about 1 day to about 14 days following administration of the hydrogel matrix to the subject. That is, the viral vector can be introduced into the hydrogel in vivo. For example, the viral vector can be introduced during gelation of the hydrogel; after gelation, but prior to administration of the hydrogel to the subject; or 1, 2, 3, 4, 5, 6, 7, 8, 9, 10, 11, 12, 13, 14, 15, 16, 17, 18, 19, 20, or 21 days after the hydrogel is administered to the subject.

It is understood and herein contemplated that the disclosed chemoattractants will leach out of or be released from the hydrogel over time and attract immune cells to the hydrogel where said immune cells (such as, for example, a T cell, NK cell, NK T cell, dendritic cell, macrophage, TINK, TIL, or MIL) can be transduced with a transgene such as, for example, a chimeric antigen receptor, NK T cell receptor, NK cell receptor (including, but not limited to anti-CD3 antibody, CD1d, an Fc fragment of an immunoglobulin, or anti-Fc gamma receptor (FcγRIII) antibody), or T cell receptor (including, but not limited to, antigen-specific T cell receptor). In one aspect, the release of the chemoattractant can be from 1 hour following administration of the hydrgogel to about 12 weeks following administration of the hydrogel. For example, the release of the chemoattractant can be from about 1, 2, 3, 4, 5, 6, 7, 8, 9, 10, 11, 12, 13, 14, 15, 16, 17, 18, 19, 20, 21, 22, 23, 24, 28, 30, 36, 42, 48, 60, 72 hours. 4, 5, 6, 7, 8, 9, 10, 11, 12, 13, 14, 15, 16, 17, 18, 19, 20, 21, 28, 30, 31 days, 5, 6, 7, 8, 9, 10, 11, or 12 weeks following administration of the hydrogel. Thus, in one aspect, disclosed herein are method of transducing an immune cell, wherein the one or more chemoattractants are released from about 1 hour after administration of the hydrogel to about 12 weeks after administration of the hydrogel. Release times of the chemoattract can be modified by applying the chemoattract to the hydrogel polymer before or after gelation where application after gelation results in faster release times and application of the chemoattractant to the polymer during or before gelation results in slower release.

In one aspect, it is recognized that the immune cells can benefit from additional stimuli to maintain the cells or activate said immune cells for use in a treatment. Thus, in one aspect, disclosed herein are methods of transducing, wherein the hydrogel further comprises one or more antibodies, cytokines, and/or co-stimulatory molecules which activate a T cell, NK cell, NK T cell, dendritic cell, macrophage, TINK, TIL, or MIL. For example, the antibody can comprises anti-CD28, CD3, B7-1, B7-2, anti-inducible costimulator (ICOS), ICOS ligand, anti-CD27, CD70, 4-1BBL, anti-41-BB, anti-CD40L, CD40, anti-DAP10, anti-CD30, CD30L, anti-TIM-1, anti-TIM-2, anti-TIM-3, anti-CD44, anti-NK1.1, lectin like transcript-1 (LLT-1), anti-CD137, CD48, MICA, anti-2B4, and anti-glucocorticoid-induced tumor necrosis factor receptor related protein (GITR); and the cytokine can comprise IL-2, IL-7, IL-15, IL-21, TNF-α, or IFN-γ.

### D. Method of Treating Cancer

In one aspect, disclosed herein are methods of treating, preventing, inhibiting, and/or reducing a cancer or metastasis in a subject comprising administering to the subject any of the hydrogel matrixes disclosed herein. For example, disclosed herein are methods of treating a cancer in a subject comprising administering to the subject a hydrogel matrix comprising one or more chemoattractants, wherein the one or more chemoattractants comprise C-C motif chemokine ligand (CCL) 1 (CCL1), CCL5, CCL19, CCL21, CCL22, CCL28, C-X-C Motif Chemokine Ligand (CXCL) 1 (CXCL1), CXCL9, CXCL10, CXCL11, or CXCL CXCL12, M-CSF, GM-CSF, MCP-1, MCP-3, CCL2, CCL3, CCL7, CCL20, CX3CL1, BRAK, IL-12, SIP, and/or MCP212; and wherein the chemoattractant attracts and retains an immune cell (such as, for example, a T cell, NK cell, NK T cell, dendritic cell, macrophage, TINK, TIL, or MIL) to the hydrogel.

In one aspect, the hydrogel matrixes used in the methods of treating, preventing, inhibiting, and/or reducing a cancer or metastasis disclosed herein can further comprises a viral vector encoding a chimeric antigen receptor (CAR), NK cell receptor (including, but not limited to anti-CD3 antibody, CD1d, an Fc fragment of an immunoglobulin, or anti-Fc gamma receptor (FcγRIII) antibody), NK T cell receptor, or T cell receptor (TCR)(including, but not limited to antigen-specific T cell receptor). In one aspect, the viral vector transduces the immune cell; and the transduced immune cell is released from the hydrogel to the cancer. It is understood and herein contemplated that the viral vector can be introduced into the hydrogel matrix prior to administration to the subject or from about 1 day to about 14 days following administration of the hydrogel matrix to the subject. That is, the viral vector can be introduced into the hydrogel in vivo. For example, the viral vector can be introduced during gelation of the hydrogel; after gelation, but prior to administration of the hydrogel to the subject; or 1, 2, 3, 4, 5, 6, 7, 8, 9, 10, 11, 12, 13, 14, 15, 16, 17, 18, 19, 20, or 21 days after the hydrogel is administered to the subject.

It is understood and herein contemplated that the disclosed chemoattractants will leach out of or be released from the hydrogel over time and attract immune cells to the hydrogel where said immune cells (such as, for example, a T cell, NK cell, NK T cell, dendritic cell, macrophage, TINK, TIL, or MIL) can be transduced with a transgene such as, for example, a chimeric antigen receptor, T cell receptor, NK cell receptor, and/or NK T cell receptor. In one aspect, the release of the chemoattractant can be from 1 hour following administration of the hydrgogel to about 12 weeks following administration of the hydrogel. For example, the release of the chemoattractant can be from about 1, 2, 3, 4, 5, 6, 7, 8, 9, 10, 11, 12, 13, 14, 15, 16, 17, 18, 19, 20, 21, 22, 23, 24, 28, 30, 36, 42, 48, 60, 72 hours, 4, 5, 6, 7, 8, 9, 10, 11, 12, 13, 14, 15, 16, 17, 18, 19, 20, 21, 28, 30, 31 days, 5, 6, 7, 8, 9, 10, 11, or 12 weeks following administration of the hydrogel. Thus, in one aspect, disclosed herein are method of treating, preventing, inhibiting, and/or reducing a cancer or metastasis, wherein the one or more chemoattractants are released from about 1 hour after administration of the hydrogel to about 12 weeks after administration of the hydrogel. Release times of the chemoattract can be modified by applying the chemoattract to the hydrogel polymer before or after gelation where application after gelation results in faster release times and application of the chemoattractant to the polymer during or before gelation results in slower release.

Similarly, it is understood and herein contemplated that one advantage of the disclosed hydrogels over traditional CAR therapy is the slow release of CAR T cells, CAR NK cells, CAR NK T cells, CARMAs, TINKs, MILs, or TILs into the tumor microenvironment. It is understood and herein contemplated that the release of the immune cells (such as, for example, a T cell, NK cell, NK T cell, dendritic cell, macrophage, TINKs, TIL, or MIL) can be from 1 hour following administration of the hydrgogel to about 12 weeks following administration of the hydrogel. For example, the release of the chemoattractant can be from about 1, 2, 3, 4, 5, 6, 7, 8, 9, 10, 11, 12, 13, 14, 15, 16, 17, 18, 19, 20, 21, 22, 23, 24, 28, 30, 36, 42, 48, 60, 72 hours, 4, 5, 6, 7, 8, 9, 10, 11, 12, 13, 14, 15, 16, 17, 18, 19, 20, 21, 28, 30, 31 days, 5, 6, 7, 8, 9, 10, 11, or 12 weeks following administration of the hydrogel. Thus, in one aspect, disclosed herein are method of treating, preventing, inhibiting, and/or reducing a cancer or metastasis, wherein the immune cell is released from about 1 week to about 12 weeks after administration of the hydrogel.

It is understood and herein contemplated that the disclosed hydrogel matrixes used in the disclosed methods of treating, preventing, inhibiting, and/or reducing a cancer or metastasis can further comprise one or more immune blockade inhibitors and/or chemotherapeutic agents. Chemotherapeutic agents that can be used in the disclosed hydrogel matrixes can comprise any chemotherapeutic known in the art, the including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane),Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine 1131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPDX, Carac (Fluorouracil—Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNIS ONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil—Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil—Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil—Topical), Fluorouracil Injection, Fluorouracil—Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil—Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). Checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

The disclosed compositions can be used to treat any disease where uncontrolled cellular proliferation occurs such as cancers. A representative but non-limiting list of cancers that the disclosed compositions can be used to treat is the following: lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, or pancreatic cancer.

### E. Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: Making Microporous Gels Via Cryogelation

Alginate is crosslinked with calcium gluconate and frozen at −20° C. Gelation occurs around the ice crystals and these ice crystals serve as porogens (FIG. 2). The macroporosity of the scaffold promotes chemokine-mediated T cell infiltration and provides the interface for transduction by viral particles. FIG. 3 shows scanning electron micrographs of gels prepared in this manner.

### 2. Example 2: Mediated Recruitment and Reprogramming of Host T Cells

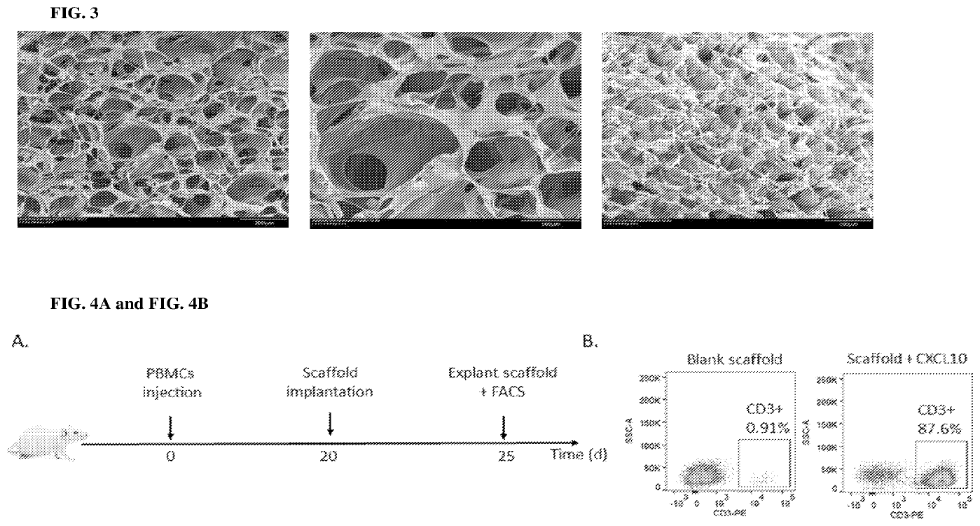

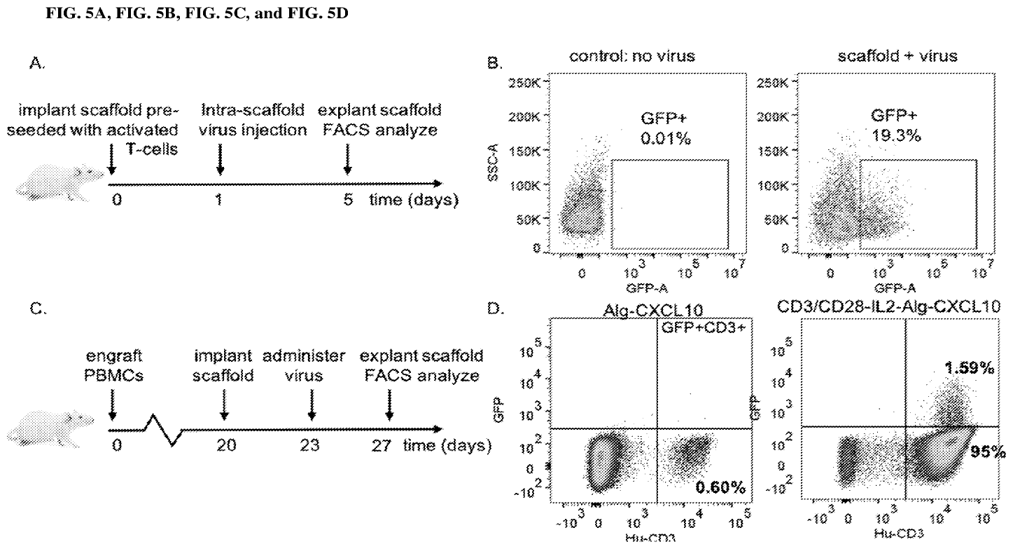

It is demonstrated herein that implanted CXCL10-releasing macroporous scaffolds efficiently recruit engrafted T cells (FIGS. 4A and 4B). Also demonstrated herein is that viral vectors transduce approximately ˜20% of activated human T cells preloaded on alginate scaffolds. (FIGS. 5A and 5B), as well as 1-2% of the 2-3 million engrafted human PBMC recruited from the circulation when combined with CD3/CD28 antibodies and IL-2 inside the gel (FIGS. 5C and 5D). the CD3/CD28 antibodies and IL-2 are provided the environmental factors to promote the activation of the T cells.

### 3. Example 3: Optimize Parameters for Scaffold-Mediated Reprogramming of T Cells

The results demonstrate that controlled release of the chemoattractant CXCL10 leads to recruitment of circulating T cells to implant scaffolds. Additionally, it is shown herein that scaffold preloaded with activated T cells can be virally transduced. The efficiency of viral vectors to transduce recruited T cells in vivo with GFP and CAR-T vectors.

### 4. Example 4: Optimization and Characterization of In Vivo Transduction of T Cells by Viral Vectors Encoding GFP

The ability of encapsulated retroviral vectors to transduce recruited T-cells can be tested. Human PBMCs in NSG mice (1×10⁷ cells/mice), can be engrafted for 20 days. Alginate scaffolds loaded with CXCL10 (4 μg/mg alginate) and GFP-encoding retroviral vectors (two different concentrations) can be implanted in the subcutaneous space of mice (n=4). At various time points (Day 4, 6, 8 after scaffold implantation), the scaffolds can be explanted, and the frequency of CD45+CD3+GFP+ cells can be assessed by flow cytometry. Controls can include: 1) blank gels without chemokine and 2) sham virus administration.

### 5. Example 5: In Situ Generation of CAR-T Cells

CAR-transduction can be accomplished in situ using chemokine loaded scaffold and CAR encoding viral vectors. Optimal recruitment and reprogramming conditions for T-cells can be tested with a CAR-encoding viral construct. n=10 can be used (see Stats. section). Scaffolds can be explanted, cells recruited and percentage of CD19. CAR+ cells can be assessed by flow cytometry. Controls can include: 1) blank gels without chemokine and 2) sham virus administration. It is possible that viral vectors can degrade over time in vivo. In such a scenario, the virus can be administered through one or multiple slow, intra-scaffold infusions. Similar results are observed from GFP- and CAR-encoding viral vectors.

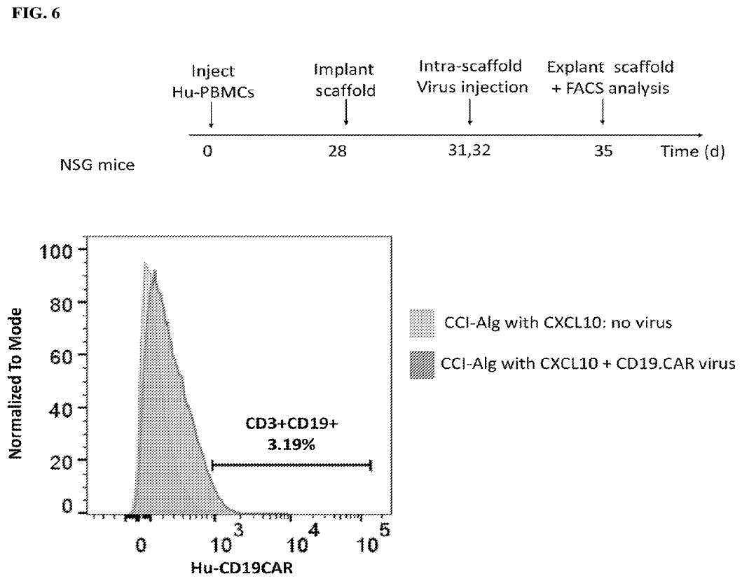

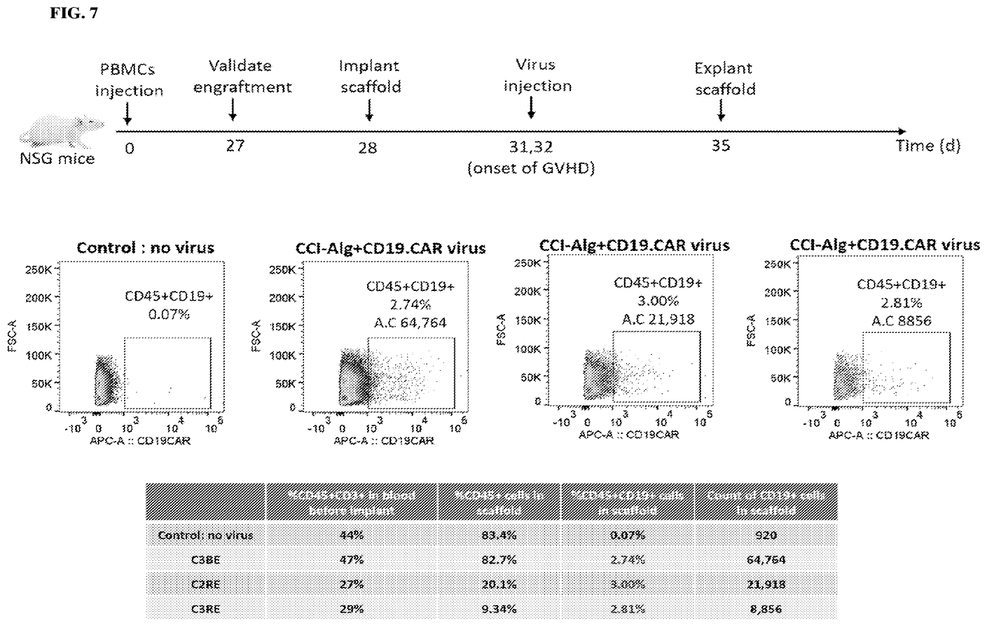

Hu-PBMC-NSG mice were implanted with scaffolds (CCI-Alg) with CXCL10. Human CD19.CAR encoded gamma retrovirus was administered by slow intra-scaffold injection. Three days post virus administration, the scaffolds were explanted, digested and isolated cells were stained with CD3 and CD19 antibody to analyze by flow cytometry (FIG. 6). FIG. 7 shows in vivo transduction of recruited T cells with CD19 CAR virus.

### 6. Example 6: Characterization of Migration of Transduced T Cells

After T cell recruitment and reprogramming, efficient release of reprogrammed cells into the circulation is crucial to therapeutic function. The efficiency and kinetics of transduced T cell migration into the systemic circulation can be characterized.

To evaluate migration of transduced T cells into the bloodstream, GFP+ or CAR+ T cells can be measured in mice (n=12) carrying scaffolds for recruitment and transduction of T-cells. Mice can be sacrificed at various time points (day 5, 10, 15 after scaffold implantation) and GFP or CAR expression in systemic T cells quantified. Best conditions can be used for T-cell recruitment and reprogramming. At different time points, T cells can be isolated from blood, spleen, bone marrow, draining and peripheral lymph nodes and the frequency and number of CD45+CD3+GFP+ or CD45+CD3+CAR+ cells assessed by flow cytometry. Controls can include: 1) scaffolds lacking virus 2) scaffolds pre-seeded with GFP+ T-cells. The macroporous nature of the scaffolds promote migration of CAR-T cells out of the scaffold and into the bloodstream. If the migration is slow or if T cells are trapped inside the scaffold, the scaffold can be modified with collagen mimicking peptides as lymphocytes are known to migrate along collagen fibers. Additionally, alginate can be chemically modified to carefully tune biodegradation rates and degradation of scaffold can be used to increase CAR-T release.

### 7. Example 7: Statistical Analysis

Animal numbers for GFP experiments were determined based on 80% power to detect above difference at 2-sided alpha level 0.05. Animal numbers for in situ generation of CAR T cells were determined based on 2-sided 95% confidence interval (CI) for the mean percentage of CD19 with half width of the CI about 3.6% with an assumed sd at about 5%.

### 8. Example 8: Tumor Model

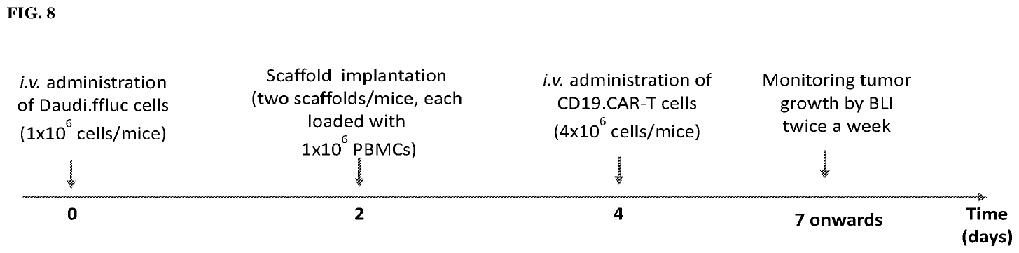

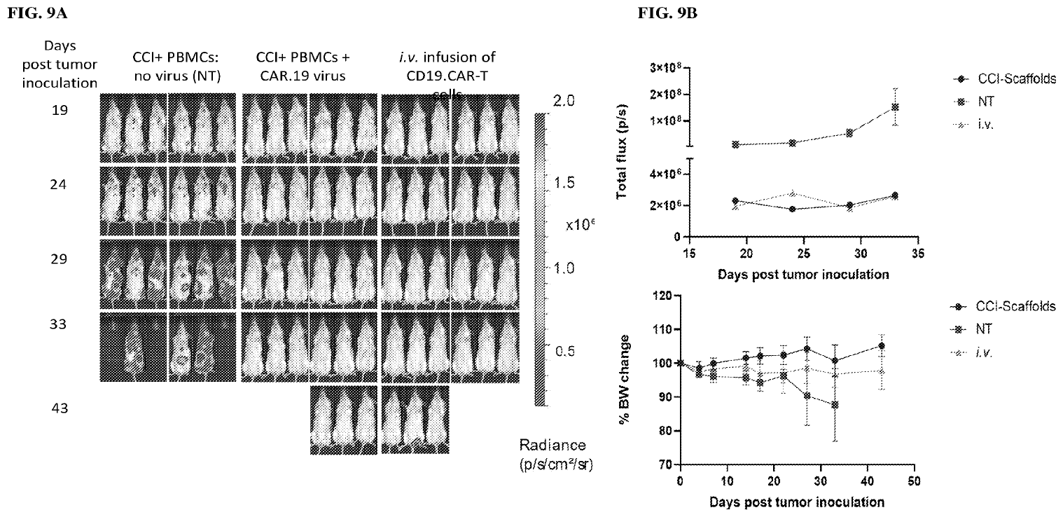

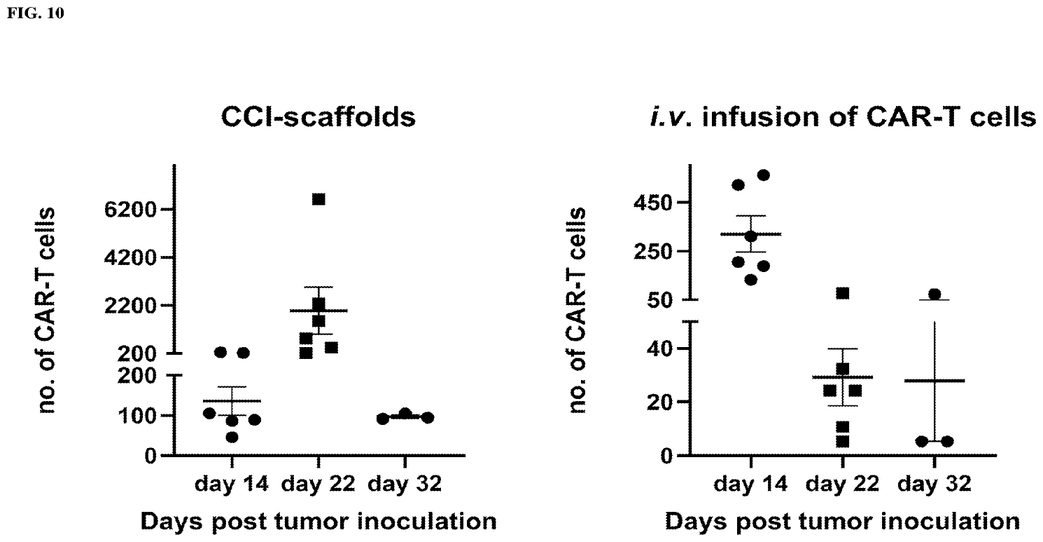

NSG mice were used to develop a mouse model for human Burkitt lymphoma (a type of B-cell lymphoma) by using the Daudi.ffluc cells (CD19+ tumor cells) (FIG. 8). Mice were monitored at 19, 24, 29, 33, and 43 days post tumor inoculation. As shown in FIG. 9A, tumor size in the mice were observed by luminescence and compared between nontreated controls and mice receiving treatment using alginate scaffolds or i.v. Mice were also measured for weight gain and total flux (FIG. 9B) showing no observable difference between the treated groups. Additionally, the number of CAR-T cells at various time points post tumor inoculation in animals receiving CD19 CAR T cells i.v. or CCI-alginate scaffolds (CCI-Alg). per 100 ul of blood in mice implanted with CCI scaffolds or i. v. infused with CAR-T cells. Mice were bled via cheek bleed, red blood cells were lysed, cells were stained with Hu-CD45, Hu-CD3 and CAR.19 antibodies and analyzed by flow cytometry (FIG. 10).

### F. References

Brentjens, R. J. et al. CD19-targeted T cells rapidly induce molecular remissions in adults with chemotherapy-refractory acute lymphoblastic leukemia. Sci. Transl. Med. 5, 177ra38 (2013).
Dotti, G., Gottschalk, S., Savoldo, B. & Brenner, M. K. Design and development of therapies using chimeric antigen receptor-expressing T cells. Immunol. Rev. 257, 107-126 (2014).
Enblad, G. et al. A PHASE I/IIa TRIAL USING CD19-TARGETED THIRD GENERATION CART CELLS FOR LYMPHOMA AND LEUKEMIA. Clin. Cancer Res. (2018). doi:10.1158/1078-0432.CCR-18-0426
Grupp, S. A. et al. Chimeric Antigen Receptor-Modified T Cells for Acute Lymphoid Leukemia. N. Engl. J. Med. 368, 1509-1518 (2013).
Kalos, M. et al. T cells with chimeric antigen receptors have potent antitumor effects and can establish memory in patients with advanced leukemia. Sci. Transl. Med. 3, 95ra73 (2011).
Kochenderfer, J. N. & Rosenberg, S. A. Treating B-cell cancer with T cells expressing anti-CD19 chimeric antigen receptors. Nat. Rev. Clin. Oncol. 10, 267-276 (2013).
Kochenderfer, J. N. et al. Eradication of B-lineage cells and regression of lymphoma in a patient treated with autologous T cells genetically engineered to recognize CD19. Blood 116, 4099-4102 (2010).
Lee, D. W. et al. T cells expressing CD19 chimeric antigen receptors for acute lymphoblastic leukaemia in children and young adults: a phase 1 dose-escalation trial. Lancet 385, 517-528 (2015).
Maude, S. L. et al. Chimeric antigen receptor T cells for sustained remissions in leukemia. N. Engl. J. Med. 371, 1507-1517 (2014).
Prasad, V. Immunotherapy: Tisagenlecleucel—the first approved CAR-T-cell therapy: implications for payers and policy makers. Nat. Rev. Clin. Oncol. 15, 11-12 (2018).
Ramos, C. A., Savoldo, B. & Dotti, G. CD19-CAR trials. Cancer J. 20, 112-118 (2014).
Sadelain, M., Brentjens, R. & Riviere, I. The promise and potential pitfalls of chimeric antigen receptors. Curr. Opin. Immunol. 21, 215-223 (2009).
Sharma, P., King, G. T., Shinde, S. S., Purev, E. & Jimeno, A. Axicabtagene ciloleucel for the treatment of relapsed/refractory B-cell non-Hodgkin's lymphomas. Drugs Today 54, 187-198 (2018).

1. A hydrogel matrix comprising one or more chemoattractants, wherein the one or more chemoattractants comprise C-C motif chemokine ligand (CCL) 1 (CCL1), CCL5, CCL19, CCL21, CCL22, CCL28, C-X-C Motif Chemokine Ligand (CXCL) 1 (CXCL1), CXCL9, CXCL10, CXCL11, CXCL12, M-CSF, GM-CSF, MCP-1, MCP-3, CCL2, CCL3, CCL7, CCL20, CX3CL1, BRAK, IL-12, S1P, and/or MCP2, wherein the chemoattractant attracts and retains an immune cell to the hydrogel.

2. The hydrogel matrix of claim 1, further comprising a viral vector encoding a chimeric antigen receptor (CAR), NK cell receptor, NK T cell receptor, or T cell receptor.

3. (canceled)

4. The hydrogel matrix of claim 1, further comprising one or more antibodies, cytokines, and/or co-stimulatory molecules which activate a T cell, macrophage, natural killer (NK) cell, NK T cell, tumor infiltrating NK cell (TINK), tumor infiltrating lymphocyte (TIL), or a marrow infiltrating lymphocyte (MIL).

5. The hydrogel matrix of claim 4, wherein the antibody comprises anti-CD3, CD28, B7-1, B7-2, anti-inducible costimulator (ICOS), ICOS ligand, anti-CD27, CD70, 4-1BBL, anti-41-BB, anti-CD40L, CD40, anti-DAP10, anti-CD30, CD30L, anti-TIM-1, anti-TIM-2, anti-TIM-3, anti-CD44, anti-NK1.1, lectin like transcript-1 (LLT-1), anti-CD137, CD48, MICA, anti-2B4, and anti-glucocorticoid-induced tumor necrosis factor receptor related protein (GITR) and wherein the cytokine comprises IL-2, IL-7, IL-15, IL-21, TNF-α, or IFN-γ.

6. (canceled)

7. The hydrogel matrix of claim 1 further comprising a chemotherapeutic agent or an immune blockade inhibitor.

8. (canceled)

9. A method of treating a cancer in a subject comprising administering to the subject a hydrogel matrix comprising one or more chemoattractants, wherein the one or more chemoattractants comprise C-C motif chemokine ligand (CCL) 1 (CCL1), CCL5, CCL19, CCL21, CCL22, CCL28, C-X-C Motif Chemokine Ligand (CXCL) 1 (CXCL1), CXCL9, CXCL10, CXCL11, CXCL12, M-CSF, GM-CSF, MCP-1, MCP-3, CCL2, CCL3, CCL7, CCL20, CX3CL1, BRAK, IL-12, S1P, and/or MCP2; and wherein the chemoattractant attracts and retains an immune cell to the hydrogel.

10. The method treating a cancer of claim 9, wherein the immune cell comprises a T cell, NK cell, NK T cell, macrophage, dendritic cell, TINK, TIL, or MTh.

11. The method treating a cancer of claim 9, wherein the hyrodrogel matrix further comprises an immune blockade inhibitor and/or a chemotherapeutic agent.

12. (canceled)

13. The method treating a cancer of claim 9, wherein the hydrogel further comprises a viral vector encoding a chimeric antigen receptor (CAR), NK cell receptor, NK T cell receptor, or T cell receptor.

14. The method treating a cancer of claim 13, wherein the viral vector transduces the immune cell; and wherein the transduced immune cell is released from the hydrogel to the cancer.

15. The method of treating a cancer of claim 14, wherein the viral vector is introduced into the hydrogel in vivo from about 1 day to about 14 days following administration of the hydrogel to the subject or wherein the viral vector is introduced into the hydrogel prior to administration of the hydrogel to the subject.

16. (canceled)

17. The method treating a cancer of claim 14 wherein the immune cell is released from about 1 week to about 12 weeks after administration of the hydrogel.

18. The method treating a cancer of claim 9, wherein the one or more chemoattractants are released from about 1 hour after administration of the hydrogel to about 12 weeks after administration of the hydrogel.

19. A method of transducing an immune cell in a subject, the method comprising administering to the subject a hydrogel comprising one or more chemoattractants and a viral vector encoding a transgene.

20. The method of transducing an immune cell of claim 19, wherein the viral vector is introduced into the hydrogel in vivo from about 1 day to about 14 days following administration of the hydrogel to the subject or the viral vector is introduced into the hydrogel prior to administration of the hydrogel to the subject.

21. (canceled)

22. The method of transducing an immune cell of claim 19, wherein the one or more chemoattractants comprise C-C motif chemokine ligand (CCL) 1 (CCL1), CCL5, CCL19, CCL21, CCL22, CCL28, C-X-C Motif Chemokine Ligand (CXCL) 1 (CXCL1), CXCL9, CXCL10, CXCL11, CXCL12, M-CSF, GM-CSF, MCP-1, MCP-3, CCL2, CCL3, CCL7, CCL20, CX3CL1, BRAK, IL-12, S1P, and/or MCP2.

23. The method of transducing an immune cell of claim 19, wherein the one or more chemoattractants are released from about 1 hour after administration of the hydrogel to about 12 weeks after administration of the hydrogel.

24. The method of transducing an immune cell of claim 19, wherein the transgene encoded by the viral vector comprises a CAR, NK cell receptor, NK T cell receptor, or T cell receptor.

25. (canceled)

26. (canceled)

27. The method of transducing an immune cell of claim 19, wherein the hydrogel further comprises one or more antibodies, cytokines, and/or co-stimulatory molecules which activate a T cell, NK cell, NK T cell, macrophage, dendritic cell, TINK, TIL, or MTh.

28. The method of transducing an immune cell of claim 27, wherein the antibody comprises anti-CD28, CD3, B7-1, B7-2, anti-inducible costimulator (ICOS), ICOS ligand, anti-CD27, CD70, 4-1BBL, anti-41-BB, anti-CD40L, CD40, anti-DAP10, anti-CD30, CD30L, anti-TIM-1, anti-TIM-2, anti-TIM-3, anti-CD44, anti-NK1.1, lectin like transcript-1 (LLT-1), anti-CD137, CD48, MICA, anti-2B4, and anti-glucocorticoid-induced tumor necrosis factor receptor related protein (GITR) and wherein the cytokine comprises IL-2, IL-7, IL-15, IL-21, TNF-α, or IFN-γ.

29. (canceled)
